(19) United States
(12) Patent Application Publication
(10) Pub. No.: US 2022/0304951 A1
(43) Pub. Date: Sep. 29, 2022

(54) ANTICANCER COMPOSITIONS AND METHODS FOR USING SAME

(71) Applicant: Lispiro LLC, Dover, MA (US)

(72) Inventor: Pavel Idelevich, Hingham, MA (US)

(21) Appl. No.: 17/706,565

(22) Filed: Mar. 28, 2022

Related U.S. Application Data
(60) Provisional application No. 63/166,609, filed on Mar. 26, 2021.

(51) Int. Cl. A61K 31/15; A61K 31/655; A61P 35/00

(52) U.S. Cl. CPC A61K 31/15; A61K 31/655; A61P 35/00

(57) ABSTRACT

In one aspect, the invention generally relates to anticancer compositions comprising new fuchsine and methods for using the anticancer compositions. In one aspect, the invention provides a method for treating a disorder of uncontrolled cellular proliferation in a cell of a subject, the method comprising the step of providing to the cell an effective amount of at least one fuchsine compound or a pharmaceutically acceptable salt thereof. This abstract is intended as a scanning tool for purposes of searching in the particular art and is not intended to be limiting of the present invention.

### CROSS REFERENCE TO RELATED APPLICATIONS

This application claims the benefit of and prior to U.S. Patent Provisional Application No. 63/166,609 filed on Mar. 26, 2021, which is herein incorporated in its entirety.

### FIELD OF INVENTION

This disclosure relates to anticancer compositions and methods for using same, and more specifically, compositions and methods comprising at least fuchsine compound.

### BACKGROUND

Disorders and diseases of uncontrolled cellular proliferation, including cancer, can affect people of all ages, including fetuses. Cancer alone is thought to be responsible for approximately 13% of all deaths worldwide. The production of cancer cells can be caused by various genetic abnormalities, with risk factors including errors in cell replication, exposure to carcinogens, such as radiation, chemicals, or infectious agents. Cancer cells are typically characterized by hyperactive growth and division, protection against programmed cell death, loss of respect for normal tissue boundaries, and the ability to thrive in a diverse range of tissues. Although cancer research remains a leading area of basic and clinical research, there still to date no cure for cancer.

Chemotherapeutic agents and radiation, which cause mutations in actively dividing cells, are intended to selectively kill the cancer cells while not effecting normal cells. Unfortunately, these cytotoxic agents, while effective in managing certain forms of cancer, are limited in their use by their adverse side effects and lack of specificity for cancer cells.

With the development of chemotherapy, survival and recovery rates of cancer patients have improved. However, anticancer agents are problematic in terms of being highly toxic and thus severely damaging to normal cells. To overcome such a side effect of anticancer agents, many recent studies have focused on developing alternative anticancer substances capable of specifically suppressing proliferation of tumor cells.

Unfortunately, however, due to the prevalence of many different types of cancers and due to the complexity of cancers, there still remains a need to develop new anti-cancer therapeutics, including the development of compounds displaying anticancer activity. This need and other needs are satisfied by the various aspects of the present disclosure.

### SUMMARY

In accordance with the purpose(s) of the invention, as embodied and broadly described herein, the invention, in one aspect, relates to anticancer methods using known compounds and pharmaceutical compositions comprising same.

Disclosed are methods for treating a disorder of uncontrolled cellular proliferation in a cell of a subject, the method comprising the step of providing to the cell an effective amount of at least one fuchsine compound or a pharmaceutically acceptable salt thereof

Also disclosed are methods for treating a disorder of uncontrolled cellular proliferation activity in a subject, the method comprising the step of providing to the subject an effective amount of at least one fuchsine compound or a pharmaceutically acceptable salt thereof, thereby preventing the cellular aging activity.

Also disclosed are methods method for the treatment of a subject, the method comprising the steps of: diagnosing the subject as having a hyperproliferative disorder or disease; and administering to the subject an effective amount of at least one fuchsine compound or a pharmaceutically acceptable salt thereof

Also disclosed are pharmaceutical compositions comprising an effective amount of at least one fuchsine compound or a pharmaceutically acceptable salt thereof; and a pharmaceutically acceptable carrier.

Also disclosed are kits comprising an effective amount of at least one fuchsine compound or a pharmaceutically acceptable salt thereof, and one or more of: a) at least one agent known to treat a hyperproliferative disorder or disease; b) instructions for treating the hyperproliferative disorder or disease; and c) instructions for administering the fuchsine compound in connection with the hyperproliferative disorder or disease.

In various further aspects, the at least one fuchsine compound can comprise a complex with at least one other compound or agent, for example, a diazo dye, such as Bismarck brown.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows stained samples of normal human colon cells in different concentration of new fuchsine in the medium.

FIG. 2 shows stained samples of human colon carcinoma cells in different concentration on new fuchsine in the medium.

FIG. 3 shows stained samples of normal human colon cells in different concentration of new fuchsine in the medium.

FIG. 4 shows stained samples of fibrosarcoma cells in different concentrations of new fuchsine in the medium.

FIG. 5 shows a graph depicting cell viability of human cervical carcinoma cell line (HeLa) after treatment using different concentration of new fuchsine.

FIG. 6 shows a graph depicting cell count of human cervical carcinoma cell line (HeLa) after treatment using different concentration of new fuchsine.

FIG. 7 shows a graph depicting cell viability of human colon adenocarcinoma cells (HT29) after treatment using different concentration of new fuchsine.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step. In some aspects of the disclosed methods, the subject has been diagnosed with a need for treatment of one or more age-related disorder or disease prior to the administering step. In some aspects of the disclosed method, the subject has been diagnosed with a chronic pulmonary disease prior to the administering step. In some aspects of the disclosed method, the subject been diagnosed with a chronic pulmonary disease prior to the administering step.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. For example, preventing age-related disease or disorder means reducing the incidences, delaying or reversing diseases or disorders that are related to or associated with aging.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of an age-related disorder or disease prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the term “providing” refers to any method of administering or contacting a disclosed compound or composition to a cell, target receptor, or other biological entity. preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “IC_{50,}” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein may comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³13 C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g., Almarasson, O., et. al. (2004) The Royal Society of Chemistry, 1889-1896. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In accordance with the purpose(s) of the invention, as embodied and broadly described herein, the invention, in one aspect, relates to fuchsine compounds and compositions useful as anticancer agents. In further aspects, the invention relates to pharmaceutical compositions comprising same, methods of treating disorders using the compounds and compositions, and methods of treating disorders associated with uncontrolled cellular proliferation.

In various aspects, the disclosed compounds and compositions have cytotoxic activity on a cell. In a further aspect, the cell is a mammalian cell. In a still further aspect, the mammalian cell is human. In a yet further aspect, the mammalian cell is murine.

In a further aspect, the disclosed compounds are cytotoxic against at least one cancer cell line. In a still further aspect, the cancer cell line is selected from HT29, HT1080, PC3, JeKo-1, JVM-2, and WIDr cells. In some aspects, the cancer cell line is a colon carcinoma cell. In a further aspect, the colon carcinoma cell line is HT29. In other aspects, the cancer cell line is a fibrosarcoma cell. In still further aspects, the fibrosarcoma cell line is HT1080. In a yet further aspect, the cancer cell line is a leukemia cell. In an even further aspect, the leukemia cell line is JeKo-1. In a still further aspect, the cancer cell line is a lymphoma cell line. In a yet further aspect, lymphoma cell line is JVM-2 (ATCC CRL-3002). In an even further aspect, the cancer cell line is a cervical cancer cell line, such as, for example, HeLa cells.

In a further aspect, the disclosed compounds have cytotoxic activity on a cell associated with uncontrolled cellular proliferation. In a yet further aspect, the cell associated with uncontrolled cellular proliferation is a cancer cell. In an even further aspect, the cell is a cell cultured in vitro. In a still further aspect, the cell is in vivo. In various aspects, the disclosed compounds disrupt or inhibit cellular processes such as cell replication.

It is contemplated that each disclosed derivative can be optionally further substituted. It is also contemplated that any one or more derivative can be optionally omitted from the invention. It is understood that the disclosed compounds can be employed in the disclosed methods of using.

In one aspect, the invention relates to fuchsine compounds or a pharmaceutically acceptable salt thereof. In a further aspect, the fuchsine compounds include compounds, such as, for example, new fuchsine ([4-[bis(4-amino-3-methylphenyl)methylidene]-2-methylcyclohexa-2,5-dien-1-ylidene]azanium; chloride), fuchsine (also referred to as rosaniline hydrochloride) (4-[(4-Aminophenyl)-(4-imino-1-cyclohexa-2,5-dienydene) methyl]aniline hydrochloride), and pararosaniline ([4-[Bis(4-aminophenyl)methylidene]-1-cyclohexa-2,5-dienylidenelazanium chloride), as well as, analogues and salts thereof. In a yet further aspect, new fuchsine refers to an organic compound with the formula RCH3N(H)CH3C6H3)3C]Cl, and can be used interchangeably with Magenta III and Basic Violet 2. It is a compound having the structure represented by the formula:

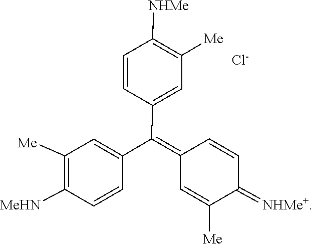

New fuchsine is commonly obtained from the condensation of N-methyltoluidine with xylidene in the presence of hydrochloric acid. In some aspects, the fuchsine compound may not comprise basic fuchsine, which is a mixture of rosaniline, pararosaniline, new fuchsine and Magenta II. In other aspects, the fuchsine compound is new fuchsine.

In further aspects, the invention relates to new fuchsine, analogues, and complexes thereof, in the form of acids, salts, esters and other derivatives. In some aspects, the invention relates to a complex of new fuchsine and a diazo dye for use as anticancer composition. In other aspects, the invention relates to a complex of new fuchsine and Bismarck brown for use as anticancer composition. In further aspects, Bismarck brown Y, also called C.I. 21000 and C.I. Basic Brown 1,[1], is a diazo dye with the formula [(H2N)2C6H3N2]2C6H4, and having a structure represented by:

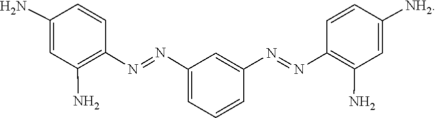 [SMILES: Nc1ccc(/N=N/c2cccc(/N=N/c3ccc(N)cc3N)c2)c(N)c1]

In still further aspects, Bismarck brown Y is a metachromatic dye which stains acid mucins yellow.

In various aspects, the disclosed compounds and compositions can have anticancer activity, and thus can be effective at treating one or more cancers and/or related disorders. In one aspect, the compound is capable of inhibiting the proliferation of at least one cell (e.g., a cancer cell). A disclosed compound can also be cytotoxic against at least one cancer cell line (e.g., a carcinoma cell line or fibrosarcoma cell line). For example, the compound can be effective at treating proliferation of human cervical carcinoma cell line (e.g., HeLa). In further aspects, a disclosed compound or composition may also be cytotoxic against a human colon carcinoma cell line such as HT29. In various further aspects, the compound or composition can be effective at treating proliferation of a human fibrosarcoma cell line such as HT-1080.

The disclosed compounds can be efficacious against a variety of cancer cell types and cell lines derived from cancer cells. If a cell line is selected from HeLa, HT29 and HT1080, for example, a compound disclosed herein can exhibit an IC₅₀ value of about 40, 50, 60, 70, 80, 90, or 100 uM against the cell line. In various aspects, the compound exhibits an IC₅₀ value of about 100 uM against a carcinoma cell line. In a further aspect, the compound exhibits an IC₅₀ value of about 100 uM against a fibrosarcoma cell line.

In various aspects, the disclosed compounds exhibit an IC₅₀ value of less than or equal to about 500 uM. In a further aspect, the disclosed compounds exhibit an IC₅₀ value of less than or equal to about 300 uM. In a still further aspect, the disclosed compounds exhibit an IC₅₀ value of less than or equal to about 100 uM. In an even further aspect, the IC₅₀ is determined using the HeLa or HT29 cell line. In a still further aspect, the IC₅₀ is determined using the HT-1080 cell line. Toxicity and therapeutic efficacy of the disclosed compounds can be determined by standard pharmaceutical procedures in cell cultures or experimental animals, e. g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices can be desirable. While compounds that exhibit toxic side effects can be used, care should be taken to design a delivery system that targets such compounds to the site of affected tissue in order to minimize potential damage to uninfected cells and, thereby, reduce side effects.

Data obtained from the cell culture assays and animal studies can be used in formulating a range of dosage for use in humans. The dosage of such compounds lies preferably within a range of circulating concentrations that include the ED₅₀ with little or no toxicity. Dosages can vary within this range depending upon the dosage form employed and the route of administration utilized. For any compound used in disclosed herein, the therapeutically effective dose can be estimated initially from cell culture assays. A dose can be formulated in animal models to achieve a circulating plasma concentration range that includes the IC₅₀ as determined in cell culture experiments. Such information can be used to more accurately determine useful doses in humans. Levels in plasma can be measured, for example, by high performance liquid chromatography.

Suitable daily doses for the treatment or prevention of a disorder described herein can be readily determined by those skilled in the art. A recommended dose of a compound of a compound disclosed herein can be from about 0.1 mg to about 1000 mg per day, e.g., from about 0.1 to about 500 mg/kg/day, 0.1 to about 250 mg/kg/day, or 0.1 to about 100 mg/kg/day, per kg of body weight, given as a single dose, a single once-a-day dose, or as divided doses throughout a selected time period.

The anti-cancer activity of the disclosed therapies can be determined by using various experimental animal models of such as the SCID mouse model or nude mice with human tumor grafts known in the art and described in Yamanaka, 2001, Microbiol Immunol 2001; 45 (7): 507-14.

The disclosed protocols and compositions can be tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed.

A lower level of proliferation or survival of the contacted cells can indicate that the therapy can be effective to treat a selected disorder in a subject. Alternatively, instead of culturing cells from a patient, protocols can be screened using cells of a tumor or malignant cell line. Many assays known in the art can be used to assess such survival and/or growth; for example, cell proliferation can be assayed by measuring 3H-thymidine incorporation, by direct cell count, by detecting changes in transcriptional activity of known genes such as proto-oncogenes or cell cycle markers; cell viability can be assessed by trypan blue staining, while differentiation can be assessed visually based on changes in morphology, etc.

Compounds for use in therapy can be tested in suitable animal model systems prior to testing in humans, including but not limited to in rats, mice, chicken, cows, monkeys, rabbits, etc. Animal models for cancer known in the art and widely used include mice, such as described in Hann et al., 2001, Curr Opin Cell Biol 2001, 13 (6): 778-84, which is incorporated herein by reference in its entirety.

Further, any assays known to those skilled in the art can be used to evaluate the prophylactic and/or therapeutic utility of the combinatorial therapies disclosed herein for treatment, prophylaxis, management or amelioration of one or more symptoms associated with the disease or disorder as described hereinabove.

### C. ANTICANCER COMPOSITIONS

In one aspect, the invention relates to compositions comprising an effective amount of at least one fuchsine compound or a pharmaceutically acceptable salt thereof. In further aspects, the at least one fuchsine compound is new fuchsine. In some aspects, the at least one fuchsine compound is not basic fuchsine.

The compounds have anticancer activity, and generally have IC₅₀ values ranging from about 0.01 micromolar to about 100 millimolar. IC₅₀ refers to the concentration of the compound that is required for 50% antagonism or inhibition of the target in vitro. IC₅₀ also refers to the concentration of a substance that is required for 50% antagonism or inhibition of the target in vivo. The activity of the compounds, including IC_{50,} is determined according to the procedures discussed below in the Examples section.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., Ansel, H. et. al., Pharmaceutical Dosage Forms and Drug Delivery Systems (6th Ed. 1995) at pp. 196 and 1456-1457.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical and nutraceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, the effective amount is a therapeutically effective amount. In a still further aspect, the effective amount is a prophylactically effective amount.

In a further aspect, the composition is formulated for parenteral administration. In a still further aspect, the composition is formulated for inhalation. In yet a further aspect, the composition is formulated for oral administration. In an even further aspect, the composition is formulated for topical administration.

It is understood that the disclosed compositions can be prepared from the disclosed compounds. It is also understood that the disclosed compositions can be employed in the disclosed methods of using.

In one aspect, the present disclosure also provides a method for making a complex of new fuchsin and a diazo dye, such as Bismarck brown. Without wishing to be bound by a particular theory, it is believed that the resulting complex of two basic stains result in a stronger cytotoxic effect. In further aspects, the complex can be prepared using equal weight parts in grams of new fuchsin and Bismarck brown powder taken and placed in glass jar, and adding distilled water in order to produce 250 micromolar concentrations. The mixture s placed on heating plate and heated to 70 degrees C. Temperature should stay stable (70 C) for 30 minutes with constant stirring. Then the mixture is allowed to cool to room temperature. And exactly the same process (heating to 70 C and steering with magnetic steerer for 30 minutes) should be repeated. Resulting solution has dark brown color. After cooling down the solution to the room temperature, it is ready to use.

### D. METHODS FOR TREATING DISEASES AND DISORDERS

In one aspect, the invention relates to methods for treating a disorder of uncontrolled cellular proliferation in a cell of a subject, the method comprising the step of providing to the cell an effective amount of at least one fuchsine compound.

In various further aspects, diseases and disorders involving uncontrolled cellular proliferation or cell hyperproliferation that can be treated or prevented include but are not limited to cancers, premalignant conditions (e.g., hyperplasia, metaplasia, and dysplasia), benign tumors, hyperproliferative disorders, and benign dysproliferative disorders. Cancer is characterized primarily by an increase in the number of abnormal cells derived from a given normal tissue, invasion of adjacent tissues by these abnormal cells, and lymphatic or blood-borne. Malignancies and related disorders that can be treated, prevented, managed, ameliorated, particularly metastatic cancer, by administration of a compound of the invention that inhibits ceramidase function as discussed below.

In one aspect, the disclosed compounds and/or compositions can be useful for the treatment of a cancer, including, but not limited to, Leukemia, acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, polycythemia vera, Lymphoma, Hodgkin's disease, non-Hodgkin's disease, multiple myeloma, Waldenstrom's macroglobulinemia, heavy chain disease, Solid tumors, sarcomas and carcinomas, fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, cancers and related disorders that can be treated or prevented by methods and compositions disclosed herein include but are not limited to the following: leukemia, including, but not limited to, acute leukemia, acute lymphocytic leukemia; acute myelocytic leukemia, including, but not limited to, myeloblastic, promyelocytic, myelomonocytic, monocytic, erythroleukemia and myelodysplastic syndrome; chronic leukemia, including, but not limited to, chronic myelocytic (granulocytic) leukemia, chronic lymphocytic leukemia, hairy cell leukemia; polycythemia vera; lymphomas, including, but not limited to, Hodgkin's lymphoma, non-Hodgkin's lymphoma; myeloma, including, but not limited, to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas, including, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumor, including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer, including, but not limited to, papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers, including, but not limited to, Cushing's disease, prolactin-secreting tumor, acromegaly, and diabetes insipius; eye cancer, including, but not limited to, ocular melanoma such as iris melanoma, choroidal melanoma, and cilliary body melanoma, and retinoblastoma; vaginal cancer, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancer, including, but not limited to, squamous cell carcinoma, and adenocarcinoma; uterine cancer, including, but not limited to, endometrial carcinoma and uterine sarcoma; ovarian cancers, including, but not limited to, ovarian epithelial carcinoma, borderline tumor, germ cell tumor, and stromal tumor; esophageal cancer, including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancer, including, but not limited to, adenocarcinoma, fungating (polypoid), ulcerating, superficial spreading, diffusely spreading, malignant lymphoma, liposarcoma, fibrosarcoma, and carcinosarcoma; colon cancer; rectal cancer; liver cancer, including, but not limited to, hepatocellular carcinoma and hepatoblastoma, gallbladder cancer, including, but not limited to, adenocarcinoma; cholangiocarcinoma, including, but not limited to, papillary, nodular, and diffuse; lung cancer, including, but not limited to, non-small cell lung cancer, squamous cell carcinoma (epidermoid carcinoma), adenocarcinoma, large-cell carcinoma and small-cell lung cancer; testicular cancer, including, but not limited to, germinal tumor, seminoma, anaplastic, classic (typical), spermatocytic, nonseminoma, embryonal carcinoma, teratoma carcinoma, and choriocarcinoma (yolk-sac tumor); prostate cancer, including, but not limited to, adenocarcinoma, leiomyosarcoma, and rhabdomyosarcoma; penal cancers; oral cancer, including, but not limited to, squamous cell carcinoma; basal cancers; salivary gland cancer, including, but not limited to, adenocarcinoma, mucoepidermoid carcinoma, and adenoidcystic carcinoma; pharynx cancer, including, but not limited to, squamous cell cancer, and verrucous; skin cancer, including, but not limited to, basal cell carcinoma, squamous cell carcinoma and melanoma, superficial spreading melanoma, nodular melanoma, lentigo malignant melanoma, and acral lentiginous melanoma; kidney cancer, including, but not limited to, renal cell cancer, adenocarcinoma, hypernephroma, fibrosarcoma, and transitional cell cancer (renal pelvis and/or ureter); Wilms' tumor; bladder cancer, including, but not limited to, transitional cell carcinoma, squamous cell cancer, adenocarcinoma, carcinosarcoma. In addition, cancer includes myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

In further aspects, the subject has been diagnosed with the disorder of uncontrolled cellular proliferation prior to the administering step. In a still further aspect, the method of treating a disorder of uncontrolled cellular proliferation in a subject further comprises the step of identifying a subject in need of treatment for a disorder of uncontrolled cellular proliferation.

In a further aspect, the step of administering occurs topically, parenterally, orally, intravenously, intramuscularly, subcutaneously or by aerosol. In a still further aspect, the step of administering occurs orally, parenterally, intramuscularly, or intravenously. In a yet further aspect, the step of administering occurs orally. In an even further aspect, step of administering occurs intravenously.

In a further aspect, the disorder of uncontrolled cellular proliferation is a cancer.

In a further aspect, the cancer is a sarcoma. In a still further aspect, the sarcoma is selected from fibrosarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, and lymphangioendotheliosarcoma.

In a further aspect, the cancer is a carcinoma. In a still further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the cancer is selected from synovioma, mesothelioma, Ewing's tumor, pancreatic cancer, breast cancer, ovarian cancer, prostate cancer, hepatoma, Wilms' tumor, cervical cancer, testicular cancer, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, the cancer is a hematological cancer. In a still further aspect, the hematological cancer is selected from a leukemia, lymphoma, chronic myeloproliferative disorder, myelodysplastic syndrome, myeloproliferative neoplasm, plasma cell neoplasm (myeloma), solid tumor, sarcoma, and carcinoma.

In a further aspect, the hematological cancer is leukemia. In a still further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia.

In a further aspect, the hematological cancer is a lymphoma. In a still further aspect, the lymphoma is selected from AIDS-Related lymphoma, cutaneous T-Cell lymphoma, Hodgkin lymphoma, non-Hodgkin lymphoma, primary central nervous system lymphoma, mycosis fungoides and the Sézary Syndrome, heavy chain disease, and Waldenstrom macroglobulinemia. In a yet further aspect, the lymphoma is selected from Hodgkin's lymphoma and non-Hodgkin's lymphoma.

In a further aspect, the lymphoma is Hodgkin's lymphoma. In a still further aspect, the Hodgkin's lymphoma is classic Hodgkin lymphoma. In a yet further aspect, the classic Hodgkin's lymphoma is selected from nodular sclerosis Hodgkin lymphoma, mixed cellularity Hodgkin lymphoma, lymphocyte-depleted Hodgkin lymphoma, and Lymphocyte-rich classic Hodgkin lymphoma. In an even further aspect, the Hodgkin's lymphoma is nodular lymphocyte-predominant lymphoma.

In a further aspect, the lymphoma is non-Hodgkin's lymphoma. In a still further aspect, the non-Hodgkin's lymphoma is a B-cell type. In a yet further aspect, the B-cell type non-Hodgkin's lymphoma is selected from Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. In an even further aspect, the non-Hodgkin's lymphoma is a T-cell type. In a still further aspect, the T-cell type non-Hodgkin's lymphoma is selected from mycosis fungoides and the Sézary Syndrome, anaplastic large cell lymphoma, and precursor T-lymphoblastic lymphoma. In a yet further aspect, the non-Hodgkin's lymphoma is a NK-cell type.

In a further aspect, the hematological cancer is a chronic myeloproliferative disorder. In a still further aspect, the chronic myeloproliferative disorder is selected from chronic myelogenous leukemia, polycythemia vera, primary myelofibrosis, chronic idiopathic myelofibrosis, essential thrombocythemia, chronic neutrophilic leukemia, and chronic eosinophilic leukemia.

In a further aspect, the hematological cancer is a myeloplastic syndrome. In a still further aspect, the myeloplastic syndrome is selected from refractory anemia (RA), refractory anemia with ring sideroblasts (RARS), refractory anemia with excess blasts (RAEB), refractory cytopenia with multilineage dysplasia (RCMD), refractory cytopenia with unilineage dysplasia (RCUD), unclassifiable myelodysplastic syndrome (MDS-u), and myelodysplastic syndrome associated with an isolated del(5q).

In a further aspect, the hematological cancer is a myeloproliferative neoplasm. In a still further aspect, the myeloproliferative neoplasm is selected from chronic myelomonocytic leukemia (CMML), juvenile myelomonocytic leukemia (JMML), atypical chronic myeloid leukemia (aCML), and unclassifiable myelodysplastic/myeloproliferative neoplasm.

In a further aspect, the hematological cancer is a plasma cell neoplasm. In a still further aspect, the plasma cell neoplasm is selected from monoclonal gammopathy of undetermined significance (MGUS), isolated plasmacytoma of the bone, extramedullary plasmacytoma, and multiple myeloma.

In various aspects, the subject is a mammal. In a further aspect, the subject is a human. In a still further aspect, the subject is a mouse.

In further aspects, the invention also relates to methods for inhibiting cell replication in at least one cell of a subject, the method comprising the step of contacting the cell an effective amount of at least one fuchsine compound. In a further aspect, inhibiting is preventing. In a still further aspect, the cell is mammalian. In a yet further aspect, the mammalian cell is human. In an even further aspect, the mammalian cell is murine. In a still further aspect, the cell is a cancer cell.

In a further aspect, the cancer cell is associated with a sarcoma. In a still further aspect, the sarcoma is selected from fibrosarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, and lymphangioendotheliosarcoma.

In a further aspect, the cancer cell is associated with a carcinoma. In a still further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the cancer cell is associated with a cancer selected from synovioma, mesothelioma, Ewing's tumor, pancreatic cancer, breast cancer, ovarian cancer, prostate cancer, hepatoma, Wilms' tumor, cervical cancer, testicular cancer, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, the cancer cell is associated with a hematological cancer. In a still further aspect, the hematological cancer is selected from a leukemia, lymphoma, chronic myeloproliferative disorder, myelodysplastic syndrome, myeloproliferative neoplasm, plasma cell neoplasm (myeloma), solid tumor, sarcoma, and carcinoma.

In a further aspect, the hematological cancer is leukemia. In a still further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia.

In a further aspect, the hematological cancer is a lymphoma. In a still further aspect, the lymphoma is selected from AIDS-Related lymphoma, cutaneous T-Cell lymphoma, Hodgkin lymphoma, non-Hodgkin lymphoma, primary central nervous system lymphoma, mycosis fungoides and the Sézary Syndrome, heavy chain disease, and Waldenstrom macroglobulinemia. In a yet further aspect, the lymphoma is selected from Hodgkin's lymphoma and non-Hodgkin's lymphoma.

In a further aspect, the lymphoma is Hodgkin's lymphoma. In a still further aspect, the Hodgkin's lymphoma is classic Hodgkin lymphoma. In a yet further aspect, the classic Hodgkin's lymphoma is selected from nodular sclerosis Hodgkin lymphoma, mixed cellularity Hodgkin lymphoma, lymphocyte-depleted Hodgkin lymphoma, and Lymphocyte-rich classic Hodgkin lymphoma. In an even further aspect, the Hodgkin's lymphoma is nodular lymphocyte-predominant lymphoma.

In a further aspect, the lymphoma is non-Hodgkin's lymphoma. In a still further aspect, the non-Hodgkin's lymphoma is a B-cell type. In a yet further aspect, the B-cell type non-Hodgkin's lymphoma is selected from Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. In an even further aspect, the non-Hodgkin's lymphoma is a T-cell type. In a still further aspect, the T-cell type non-Hodgkin's lymphoma is selected from mycosis fungoides and the Sézary Syndrome, anaplastic large cell lymphoma, and precursor T-lymphoblastic lymphoma. In a yet further aspect, the non-Hodgkin's lymphoma is a NK-cell type.

In a further aspect, the hematological cancer is a chronic myeloproliferative disorder. In a still further aspect, the chronic myeloproliferative disorder is selected from chronic myelogenous leukemia, polycythemia vera, primary myelofibrosis, chronic idiopathic myelofibrosis, essential thrombocythemia, chronic neutrophilic leukemia, and chronic eosinophilic leukemia.

In a further aspect, the hematological cancer is a myeloplastic syndrome. In a still further aspect, the myeloplastic syndrome is selected from refractory anemia (RA), refractory anemia with ring sideroblasts (RARS), refractory anemia with excess blasts (RAEB), refractory cytopenia with multilineage dysplasia (RCMD), refractory cytopenia with unilineage dysplasia (RCUD), unclassifiable myelodysplastic syndrome (MDS-u), and myelodysplastic syndrome associated with an isolated del(5q).

In a further aspect, the hematological cancer is a myeloproliferative neoplasm. In a still further aspect, the myeloproliferative neoplasm is selected from chronic myelomonocytic leukemia (CMML), juvenile myelomonocytic leukemia (JMML), atypical chronic myeloid leukemia (aCML), and unclassifiable myelodysplastic/myeloproliferative neoplasm.

In a further aspect, the hematological cancer is a plasma cell neoplasm. In a still further aspect, the plasma cell neoplasm is selected from monoclonal gammopathy of undetermined significance (MGUS), isolated plasmacytoma of the bone, extramedullary plasmacytoma, and multiple myeloma.

In a further aspect, the cell has been isolated from a human prior to the contacting step. In a still further aspect, the step of contacting occurs in vitro. In a yet further aspect, the step of contacting occurs in vivo.

In a further aspect, the step of contacting occurs via administration of the compound to a subject. In a still further aspect, the step of contacting occurs via administration of the compound to a subject, wherein the subject is a mammal. In a yet further aspect, the step of contacting occurs via administration of the compound to a subject, wherein the subject is a human. In an even further aspect, the step of contacting occurs via administration of the compound to a subject, wherein the subject is a mouse.

In a further aspect, the step of contacting occurs via administration of the compound to a subject, wherein the administration occurs topically, parenterally, orally, intravenously, intramuscularly, subcutaneously, or by aerosol. In a still further aspect, the step of contacting occurs via administration of the compound to a subject, wherein the administration occurs orally, parenterally, intramuscularly, or intravenously. In a yet further aspect, the step of contacting occurs via administration of the compound to a subject, wherein the administration occurs orally. In an even further aspect, the step of contacting occurs via administration of the compound to a subject, wherein the administration occurs intravenously.

In a further aspect, the step of contacting occurs via administration of the compound to a subject, wherein the subject has been diagnosed with a need for inhibiting cell replication prior to the administering step. In a still further aspect, the step of contacting occurs via administration of the compound to a subject, wherein the subject has been diagnosed with a need for treatment of a disorder of uncontrolled cellular proliferation prior to the administering step.

To treat or control the cellular hyperproliferative or cancer activity, the compounds and pharmaceutical compositions comprising the compounds are provided to a cell, such as a mammalian cell, e.g., a human cell. In a further aspect, providing can comprise administering or contacting the cell with the compounds or compositions. In a still further aspect, prior to providing the compounds or compositions, the cell can be identified with a need for treatment of anticancer treatment, as described herein.

In some aspects, the fuchsine compound can be used at low doses. In a further aspect, the fuchsine compound dose can be from about 0.001 to about 10000 μM in serum medium. In a still further aspect, the effective amount of the fuchsine compound is from about 0.01 to about 100 μM in serum medium, including exemplary subranges of about 0.1 to about 10, and about 0.2 to about 1 μM. In an even further aspect, the effective amount of the fuchsine compound is from about 0.1 to about 1000 μM in serum medium, including exemplary subranges of about 1 to about 250 μM, about 1 to about 150 μM, about 1 to about 125 μM, about 1 to about 100 μM, about 1 to about 62.5 μM, about 1 to about 50 μM, about 1 to about 31 μM, and about 1 to about 15 μM.

In other aspects, the invention also relates to methods for treating a disorder of uncontrolled cellular proliferation activity in a subject, the method comprising the step of providing to the subject an effective amount of at least one intracellular alkalinizing agent or a pharmaceutically acceptable salt thereof, thereby preventing the uncontrolled cellular proliferation activity.

In various aspects, it has been surprising discovered that new fuchsine compound exhibits the disclosed novel anticancer activity. Without wishing to be bound by a particular theory, it is believed the new fuchsine compound beneficially utilizes increased intracellular pH in vivo to allow binding with proteins, such as cancer cells. In general terms, as the intracellular pH increases, deprotonation of the acidic and basic groups on proteins occur, so that carboxyl groups are converted to carboxylate anions (R—COOH to R—COO—) and ammonium groups are converted to amino groups (R—NH3+ to R—NH2). In proteins, the isoelectric point (pI) is defined as the pH at which a protein has no net charge. When the pH>pI, a protein has a net negative charge and when the pH<pI, a protein has a net positive charge. The pI varies for different proteins.

Since proteins are mostly negatively charged in high pH, they are believed to bind strongly with fuchsine molecule, which is positive charged. Upon binding to the protein or cell, cell function is disrupted and the cell dies. In normal cells, where intracellular pH is neutral or slightly acidic vs neoplastic cells, proteins are mostly positive charged and, thus would not generally react with the positively charged new fuchsine molecule. This allows lower concentrations of new fuchsine to be toxic for cancer cells, yet non-toxic for normal cells.

To treat or control the hyperproliferative disease or disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human.

In a further aspect, the subject has been diagnosed with a need for anticancer treatment prior to the administering step. In a still further aspect, the subject has been diagnosed with a hyperproliferative disorder or disease. Prior to administering the compounds or compositions, the subject can be diagnosed with a need for treatment of a hyperproliferative disorder or disease, such as cancer. In a still further aspect, the subject can be identified with a need for treatment of anticancer treatment, as described herein.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disease or condition.

The effective amount or dosage of the compounds can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 1 mg to about 10,000 mg, preferably from about 2 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days. In some aspects, the compounds can be administered on a regimen of 1 to 4 times per day, preferably once or twice per day. This dosing regimen can be adjusted to provide the optimal therapeutic response.

Disclosed are methods of treating a hyperproliferative disorder in a mammal, comprising the step of administering to the mammal an effective amount of at least one of the disclosed compounds or compositions.

Disclosed are methods of inducing apoptosis in a mammal, comprising the step of administering to the mammal an effective amount of at least one of the disclosed compounds or compositions.

Disclosed are methods of inhibiting various aspects of cell function and activity, for example, and without limitation, enzymatic activity, genome and mitochondrial functions, in a mammal, comprising the step of administering to the mammal an effective amount of at least one of the disclosed compounds or compositions.

Disclosed are methods for suppressing tumor activity in a mammal, comprising the step of administering to the mammal an effective amount of at least one of the disclosed compounds or compositions.

In a further aspect, the fuchsine compound is also used at low doses in a subject. In a further aspect, the fuchsine compound dose is from about 0.1 mg to about 10,000 mg in a subject. In a still further aspect, the effective amount of the fuchsine compound is from about 0.1 mg to about 100 mg in a subject, including exemplary subranges of about 1 to about 50 mg, and about 2 to about 25 mg. In an even further aspect, the effective amount of the fuchsine compound is from about 1 to about 1,000 mg in a subject, including exemplary subranges of about 1 to about 100 mg, and about 1 to about 50 mg, including dose therebetween, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49 mg.

In determining the effective dose or dosage of the pharmaceutical composition of the invention, a response to a prophylactic and/or treatment method of the invention can, for example, also be measured by determining the physiological effects of the treatment or medication, such as the decrease or lack of disease symptoms following administration of the treatment or pharmacological agent. Other assays will be known to one of ordinary skill in the art and can be employed for measuring the level of the response. For example, the diagnostic methods that are used to ascertain the likelihood that a subject has an age-related disorder or disease can be used to ascertain the level of response to a prophylactic and/or treatment method of the invention. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount can depend upon the degree to which an individual has abnormal levels and/or activity of a pathway associated protein or pathway associated protein complex.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

In some aspects, the effective amount is a therapeutically effective amount. In other aspects, the effective amount is a prophylactically effective amount. In further aspects, the subject is a mammal. In still further aspects, the mammal is a human.

In a further aspect, the method further comprises the step of identifying a subject in need of anticancer treatment. In a still further aspect, the subject in need of anticancer treatment comprises having at least one risk factor for developing a hyperproliferative disorder or disease.

In another aspect, a method of diagnosis comprises performing an experiment upon the subject and identifying a level of a biological marker. In a further aspect diagnosing comprises determining, in a patient, levels of a marker (e.g., gene expression) indicative of a state of the patient, the state being predictive as to whether the patient will manifest reduced symptoms in response to a treatment.

In a further aspect, the biological marker is a marker for a hyperproliferative disorder or disease. In a still further aspect, the subject is a biological sample. In a still further aspect, the biological sample is selected from a cell, blood, saliva, urine, tissue, or phlegm.

In one aspect, diagnosis of a hyperproliferative disorder or disease comprises a medical history. In a further aspect, the diagnosis comprises comparing the findings of the medical history with the diagnostic standards. In a still further aspect, the diagnosis comprises finding of at least one risk factor for developing an a hyperproliferative disorder or disease.

### E. CO-THERAPEUTIC USE

In further aspects, other cancer treatments can be used in combination with the administration of one or more compounds disclosed herein. Such treatments include the use of one or more molecules, or compounds for the treatment of cancer (i.e., cancer therapeutics). Some examples include, but are not limited to, chemo agents, immunotherapeutics, cancer vaccines, anti-angiogenic agents, cytokines, hormone therapies, gene therapies, biological therapies, and radiotherapies. While maintaining or enhancing efficacy of treatment, preferably the methods of the present invention increase patient compliance, improve therapy and/or reduce unwanted or adverse effects.

In one aspect, the methods of the invention includes the administration of one or more angiogenesis inhibitors such as but not limited to: angiostatin (plasminogen fragment); antiangiogenic antithrombin III; Angiozyme; ABT-627; Bay 12-9566; Benefin; bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2- Methoxyestradiol; MMI 270 (CGS 27023A); MoAb IMC-1C11; Neovastat; NM-3; Panzem; PI-88; Placenta ribonuclease inhibitor; Plasminogen activator inhibitor; Platelet factor-4 (PF4); Prinomastat; Prolactin 16kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; D 6474; farnesyl transferase inhibitors (FTI); and bisphosphonates.

Additional examples of anti-cancer agents that can be used in the various aspects disclosed herein, including pharmaceutical compositions and dosage forms disclosed herein, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5 -azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. These two agents are particularly useful when used in methods employing thalidomide and a topoisomerase inhibitor.

In a further aspect, the treatment methods disclosed herein includes the administration of one or more immunotherapeutic agents, such as antibodies and immunomodulators, which include, but are not limited to, HERCEPTINS, RITUXANS, OVAREX™, PANOREX@, BEC2, IMC-C225, VITAMIN, CAMPATH@ I/H, Smart MI95, LYMPHOCIDE™, Smart I D10, and ONCOLYM™, rituximab, gemtuzumab, or trastuzumab.

In a still further aspect, the treatment methods disclosed herein includes administering one or more anti-angiogenic agents, which include, but are not limited to, angiostatin, thalidomide, kringle 5, endostatin, other Serpins, anti-thrombin, 29 kDa N-terminal and 40 kDa C-terminal proteolytic fragments of fibronectin, 16 kDa proteolytic fragment of prolactin, 7.8 kDa proteolytic fragment of platelet factor-4, a 13- amino acid peptide corresponding to a fragment of platelet factor-4, a 14-amino acid peptide corresponding to a fragment of collagen I<BR><BR>, a 19 amino acid peptide corresponding to a<BR>fragment of Thrombospondin I, a 20-amino acid peptide corresponding to a fragment of SPARC, or any fragments, family members, or derivatives thereof, including pharmaceutically acceptable derivatives thereof.

In one aspect, the treatment methods disclosed herein can comprise the use of radiation.

In a further aspect, the treatment methods further comprises the administration of one or more cytokines, which includes, but is not limited to, lymphokines, tumor necrosis factors, tumor necrosis factor-like cytokines, lymphotoxin-a, lymphotoxin-b, interferon-a, interferon-b, macrophage inflammatory proteins, granulocyte monocyte colony stimulating factor, interleukins (including, but not limited to, interleukin-1, interleukin-2, interleukin-6, interleukin-12, interleukin-15, interleukin-18), OX40, CD27, CD30, CD40 or CD137 ligands, Fas-Fas ligand, 4-1BBL, endothelial monocyte activating protein or any fragments, family members, or derivatives thereof, including pharmaceutically acceptable salts thereof

In a further aspect, the treatment method comprises hormonal treatment. Hormonal therapeutic treatments comprise hormonal agonists, hormonal antagonists (e.g., flutamide, tamoxifen, leuprolide acetate (LUPRONTM), LH-RH antagonists), inhibitors of hormone biosynthesis and processing, steroids (e. g., dexamethasone, retinoids, betamethasone, cortisol, cortisone, prednisone, dehydrotestosterone, glucocorticoids, mineralocorticoids, estrogen, testosterone, progestins), antigestagens (e. g., mifepristone, onapristone), antiandrogens (e. g., cyproterone acetate), and the like.

### F. PROPHYLACTIC TREATMENT

In further aspects, the disclosed compounds, compositions, and methods can be used prophylactically, i.e., to prevent progression to a neoplastic or malignant state, including but not limited to those disorders listed herein. Such prophylactic use is indicated in conditions known or suspected of preceding progression to neoplasia or cancer, in particular, where non-neoplastic cell growth consisting of hyperplasia, metaplasia, or most particularly, dysplasia has occurred.

Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

### G. KITS

In one aspect, the invention relates to a kit comprising an effective amount of at least one fuchsine compound or a pharmaceutically acceptable salt thereof, and one or more of: a) at least one agent known to treat a disorder of uncontrolled cellular proliferation; b) instructions for treating a disorder of uncontrolled cellular proliferation; and c) instructions for administering the fuchsine compound or the at least one agent in connection with the disorder of uncontrolled cellular proliferation.

The kits can also comprise compounds co-packaged, co-formulated, and/or co-delivered with other components. For example, a drug manufacturer, a drug reseller, a physician, a compounding shop, or a pharmacist can provide a kit comprising a disclosed compound and another component for delivery to a patient.

In a further aspect, the at least one compound and the at least one agent are co-formulated. In a still further aspect, the at least one compound and the at least one agent are co-packaged.

In a further aspect, the at least one agent is a hormone therapy agent. In a still further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

In a further aspect, the at least one agent is a chemotherapeutic agent. In a still further aspect, the chemotherapeutic agent is selected from one or more of the group consisting of an alkylating agent, an antimetabolite agent, an antineoplastic antibiotic agent, a mitotic inhibitor agent, an mTor inhibitor agent or other chemotherapeutic agent.

In a further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof

In a further aspect, the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.

In a further aspect, the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.

In a further aspect, the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.

In a further aspect, the mTor inhibitor agent is selected from one or more of the group consisting of everolimus, siroliumus, and temsirolimus, or a pharmaceutically acceptable salt, hydrate, solvate, or polymorph thereof.

In a further aspect, the kit further comprises instructions to provide the compound in connection with surgery. In a still further aspect, the kit further comprises instructions to provide the compound in connection with surgery, wherein the instructions provide that surgery is performed prior to the administering of at least one compound. In a yet further aspect, the kit further comprises instructions to provide the compound in connection with surgery, wherein the instructions provide that surgery is performed after the administering of at least one compound. In an even further aspect, the kit further comprises instructions to provide the compound in connection with surgery, wherein the instructions provide that surgery is performed after the administering of at least one compound, and wherein the instructions provide that the administering of at least one compound is to effect pre-surgical debulking of a tumor. In a still further aspect, the kit further comprises instructions to provide the compound in connection with surgery, wherein the instructions provide that surgery is performed after the administering of at least one compound, and wherein the instructions provide that surgery is performed at about the same time as the administering of at least one compound.

In a further aspect, the kit further comprises instructions to provide the compound in connection with radiotherapy. In a still further aspect, the kit further comprises instructions to provide the compound in connection with radiotherapy, wherein the instructions provide that radiotherapy is performed prior to the administering of at least one compound. In a yet further aspect, the kit further comprises instructions to provide the compound in connection with radiotherapy, wherein the instructions provide that radiotherapy is performed after the step of the administering of at least one compound. In an even further aspect, the kit further comprises instructions to provide the compound in connection with radiotherapy, wherein the instructions provide that radiotherapy is performed at about the same time as the step of the administering of at least one compound.

In a further aspect, the kit further comprises a plurality of dosage forms, the plurality comprising one or more doses; wherein each dose comprises a therapeutically effective amount of the compound and the at least one agent. In a still further aspect, the kit further comprises a plurality of dosage forms, the plurality comprising one or more doses; wherein each dose comprises a therapeutically effective amount of the compound and the at least one agent, and wherein each dose of the compound and the at least one agent are co-formulated. In a yet further aspect, the kit further comprises a plurality of dosage forms, the plurality comprising one or more doses; wherein each dose comprises a therapeutically effective amount of the compound and the at least one agent, and wherein each dose of the compound and the at least one agent are co-packaged.

In a further aspect, the kit further comprises a plurality of dosage forms, the plurality comprising one or more doses; wherein each dose comprises a therapeutically effective amount of the compound and the at least one agent, and wherein the dosage forms are formulated for oral administration and/or intravenous administration. In a still further aspect, the kit further comprises a plurality of dosage forms, the plurality comprising one or more doses; wherein each dose comprises a therapeutically effective amount of the compound and the at least one agent, and wherein the dosage forms are formulated for oral administration. In a yet further aspect, the kit further comprises a plurality of dosage forms, the plurality comprising one or more doses; wherein each dose comprises a therapeutically effective amount of the compound and the at least one agent, and wherein the dosage forms are formulated for intravenous administration.

In a further aspect, the kit further comprises a plurality of dosage forms, the plurality comprising one or more doses; wherein each dose comprises a therapeutically effective amount of the compound and the at least one agent; and wherein the dosage form for the compound is formulated for oral administration and the dosage form for the at least one agent is formulated for intravenous administration. In a still further aspect, the kit further comprises a plurality of dosage forms, the plurality comprising one or more doses; wherein each dose comprises a therapeutically effective amount of the compound and the at least one agent; and wherein the dosage form for the compound is formulated for intravenous administration and the dosage form for the at least one agent is formulated for oral administration.

In a further aspect, the instructions for treating a disorder of uncontrolled cellular proliferation provide instructions for treating a hyperproliferative disorder.

In a still further aspect, the hyperproliferative disorder is selected from a malignant, pre-malignant or non-malignant neoplastic disorder, inflammation, an autoimmune disorder, a hematological disorder, a skin disorder, a virally-induced hyperproliferative disorder, a myelodysplastic disorder or a myeloproliferative disorder.

In a yet further aspect, the hyperproliferative disorder is selected from cancer, benign tumors, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), my asthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

In a further aspect, the instructions for treating a disorder of uncontrolled cellular proliferation provide instructions for treating a cancer.

In a still further aspect, the cancer is a hematological cancer.

In an even further aspect, the hematological cancer is selected from a leukemia, lymphoma, chronic myeloproliferative disorder, myelodysplastic syndrome, myeloproliferative neoplasm, and plasma cell neoplasm (myeloma).

In a further aspect, the cancer is a sarcoma. In a still further aspect, the sarcoma is selected from fibrosarcoma, myxosarcoma, liposarcoma, chondrosarcoma, osteogenic sarcoma, chordoma, angiosarcoma, endotheliosarcoma, lymphangiosarcoma, leiomyosarcoma, rhabdomyosarcoma, and lymphangioendotheliosarcoma.

In a further aspect, the cancer is a carcinoma. In a still further aspect, the carcinoma is selected from colon carcinoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, bile duct carcinoma, choriocarcinoma, seminoma, embryonal carcinoma, lung carcinoma, small cell lung carcinoma, bladder carcinoma, and epithelial carcinoma.

In a further aspect, the cancer is selected from synovioma, mesothelioma, Ewing's tumor, pancreatic cancer, breast cancer, ovarian cancer, prostate cancer, hepatoma, Wilms' tumor, cervical cancer, testicular cancer, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In a further aspect, the cancer is a hematological cancer. In a still further aspect, the hematological cancer is selected from a leukemia, lymphoma, chronic myeloproliferative disorder, myelodysplastic syndrome, myeloproliferative neoplasm, plasma cell neoplasm (myeloma), solid tumor, sarcoma, and carcinoma.

In a further aspect, the hematological cancer is leukemia. In a still further aspect, the leukemia is selected from acute leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, and chronic lymphocytic leukemia.

In a further aspect, the hematological cancer is a lymphoma. In a still further aspect, the lymphoma is selected from AIDS-Related lymphoma, cutaneous T-Cell lymphoma, Hodgkin lymphoma, non-Hodgkin lymphoma, primary central nervous system lymphoma, mycosis fungoides and the Sezary Syndrome, heavy chain disease, and Waldenstrom macroglobulinemia. In a yet further aspect, the lymphoma is selected from Hodgkin's lymphoma and non-Hodgkin's lymphoma.

In a further aspect, the lymphoma is Hodgkin's lymphoma. In a still further aspect, the Hodgkin's lymphoma is classic Hodgkin lymphoma. In a yet further aspect, the classic Hodgkin's lymphoma is selected from nodular sclerosis Hodgkin lymphoma, mixed cellularity Hodgkin lymphoma, lymphocyte-depleted Hodgkin lymphoma, and Lymphocyte-rich classic Hodgkin lymphoma. In an even further aspect, the Hodgkin's lymphoma is nodular lymphocyte-predominant lymphoma.

In a further aspect, the lymphoma is non-Hodgkin's lymphoma. In a still further aspect, the non-Hodgkin's lymphoma is a B-cell type. In a yet further aspect, the B-cell type non-Hodgkin's lymphoma is selected from Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. In an even further aspect, the non-Hodgkin's lymphoma is a T-cell type. In a still further aspect, the T-cell type non-Hodgkin's lymphoma is selected from mycosis fungoides and the Sézary Syndrome, anaplastic large cell lymphoma, and precursor T-lymphoblastic lymphoma. In a yet further aspect, the non-Hodgkin's lymphoma is a NK-cell type.

In a further aspect, the hematological cancer is a chronic myeloproliferative disorder. In a still further aspect, the chronic myeloproliferative disorder is selected from chronic myelogenous leukemia, polycythemia vera, primary myelofibrosis, chronic idiopathic myelofibrosis, essential thrombocythemia, chronic neutrophilic leukemia, and chronic eosinophilic leukemia.

In a further aspect, the hematological cancer is a myeloplastic syndrome. In a still further aspect, the myeloplastic syndrome is selected from refractory anemia (RA), refractory anemia with ring sideroblasts (RARS), refractory anemia with excess blasts (RAEB), refractory cytopenia with multilineage dysplasia (RCMD), refractory cytopenia with unilineage dysplasia (RCUD), unclassifiable myelodysplastic syndrome (MDS-u), and myelodysplastic syndrome associated with an isolated del(5q).

In a further aspect, the hematological cancer is a myeloproliferative neoplasm. In a still further aspect, the myeloproliferative neoplasm is selected from chronic myelomonocytic leukemia (CMML), juvenile myelomonocytic leukemia (JMML), atypical chronic myeloid leukemia (aCML), and unclassifiable myelodysplastic/myeloproliferative neoplasm.

In a further aspect, the hematological cancer is a plasma cell neoplasm. In a still further aspect, the plasma cell neoplasm is selected from monoclonal gammopathy of undetermined significance (MGUS), isolated plasmacytoma of the bone, extramedullary plasmacytoma, and multiple myeloma.

In a further aspect, the compounds and the at least one agent are co-packaged. In a still further aspect, the compounds and the at least one agent are co-formulated. In a yet further aspect, each dose of the fuchsine compound and the agent are co-packaged. In an even further aspect, each dose of the fuchsine compound and the at least one agent are co-formulated.

In a further aspect, plurality of dosage forms, the plurality comprising one or more doses; wherein each dose comprises an effective amount of the fuchsine compound and the at least one agent. In a still further aspect, each dose of the fuchsine compound and the at least one agent are administered sequentially. In an even further aspect, each dose of the fuchsine compound and the at least one agent are administered simultaneously.

In a still further aspect, the effective amount is a therapeutically effective amount. In yet a further aspect, the effective amount is a prophylactically effective amount.

In a further aspect, the dosage forms are formulated for oral administration, inhalation, topical administration, and/or parenteral administration. In a still further aspect, the dosage form for the compounds is formulated for oral administration. In yet a further aspect, the dosage form for the compound is formulated for oral administration and the dosage form for the at least one agent is formulated for oral administration.

It is understood that the disclosed kits can be prepared from the disclosed compounds, products, and pharmaceutical compositions. It is also understood that the disclosed kits can be employed in connection with the disclosed methods of using.

### H. SUBJECTS

The compounds, compositions, and methods disclosed herein can be useful for the treatment or prevention of one or more disorders associated with uncontrolled cellular proliferation in a subject, as discussed hereinabove. In general, a subject can be any age, including a fetus. A subject to which a compound or compositions disclosed herein can be administered can be an animal, including but not limited to a mammal, such as a non-primate mammal (e.g., cows, pigs, sheep, goats, horses, chickens, dogs, rats, etc.) and a primate (e.g., a monkey such as an acynomolgous monkey and a human). A subject can also be a laboratory animal (e.g., a mouse, rabbit, guinea pig, fruit fly, etc.).

In one aspect, a subject can be diagnosed with one or more disorders as discussed herein elsewhere. In a specific aspect, a subject can be diagnosed with one or more disorders as discussed herein elsewhere before the step of administering to the subject a therapeutically effective amount of one or more compounds disclosed herein.

In a further aspect, a subject can be a subject in need of treatment for disorder of uncontrolled cellular proliferation, e.g., cancer. In a still further aspect, a subject can have cancer or a related disorder, as discussed hereinbefore. In one aspect, a subject can be treated prophylactically with a compound or composition disclosed herein, as discussed herein elsewhere.

One or more compounds or compositions disclosed herein can be utilized for the prevention of a variety of cancers, e. g, in individuals who are predisposed as a result of familial history or in individuals with an enhanced risk to cancer due to environmental factors.

The methods and compositions of the invention can be used in patients who are treatment naive, in patients who have previously received or are currently receiving treatment with other pharmaceutical agents or combinations, including but not limited to anti-cancer agents. Other subjects can include patients that have metastasis or no metastasis.

The methods and compositions of the invention are useful not only in untreated patients but are also useful in the treatment of patients partially or completely un-responsive to other treatments. In various aspects, the disclosure provides methods and compositions useful for the treatment of diseases or disorders in patients that have been shown to be or can be refractory or non-responsive to therapies comprising the administration of other agents.

In one aspect, subjects that can be treated with the compositions disclosed herein include those subjects displaying the presence of one or more characteristics of a transformed phenotype, or of a malignant phenotype, displayed in vivo or displayed in vitro by a cell sample from a subject, can indicate the desirability of prophylactic/therapeutic administration of a compound or composition disclosed herein. As mentioned hereinabove, such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 Dalton cell surface protein, etc.

In a further aspect, a subject that exhibits one or more of the following predisposing factors for malignancy can be treated by administration of an effective amount of a compound disclosed herein: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), and a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome.

### I. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. New Fuchsine Treatment of Human Colon Carcinoma Cells (HT29) vs Normal Human Fibroblasts (CRL-1790)

Here we investigated effect of new fuchsine treatment of Human colon carcinoma cells (HT29) vs Normal Human epithelial like cells (CRL-1790).

In this Example, by different concentration of new fuchsine on normal human epithelial like cell line CRL 1790 (ATCC) and Human Colon carcinoma cell line (HT29) was studied.

The general study methods of study were as follows:

4 flasks Thermo Scientific Nunc Nunclon Delta Surface 12.5 cm (Cat.No136196) with McCoy's5A complete medium were seeded with cell culture of CRL1790 (ATCC) and 4 flasks Thermo Scientific Nunc Nunclon Delta Surface 12.5 cm (Cat.No136196) with McCoy's5A complete medium were seeded with cell culture of HT29 (ATCC) and were kept at 37 degrees 5% CO2 in CO2 incubator until it reached of 70% confluence (2 days). After reaching of 70% confluence flasks McCoy's5A medium with 10% of FBS was changed to the same medium with different concertation of new fuchsine (0 uM, 25 uM, 50 uM, 100 uM, 150 uM) (Sigma Cat. No 72200).

Flasks were kept in the CO2 incubator 5% CO2, 37-degree C for additional 48 hours. After 48 hours of treatment all flasks were stained by fixed by 10% Formaldehyde and stained by H&E.

After completing of the staining protocol the flasks were examined in the light microscope under magnification x100 and multiple pictures were taken using Olympus BX51 microscope and Olympus DP73 digital camera.

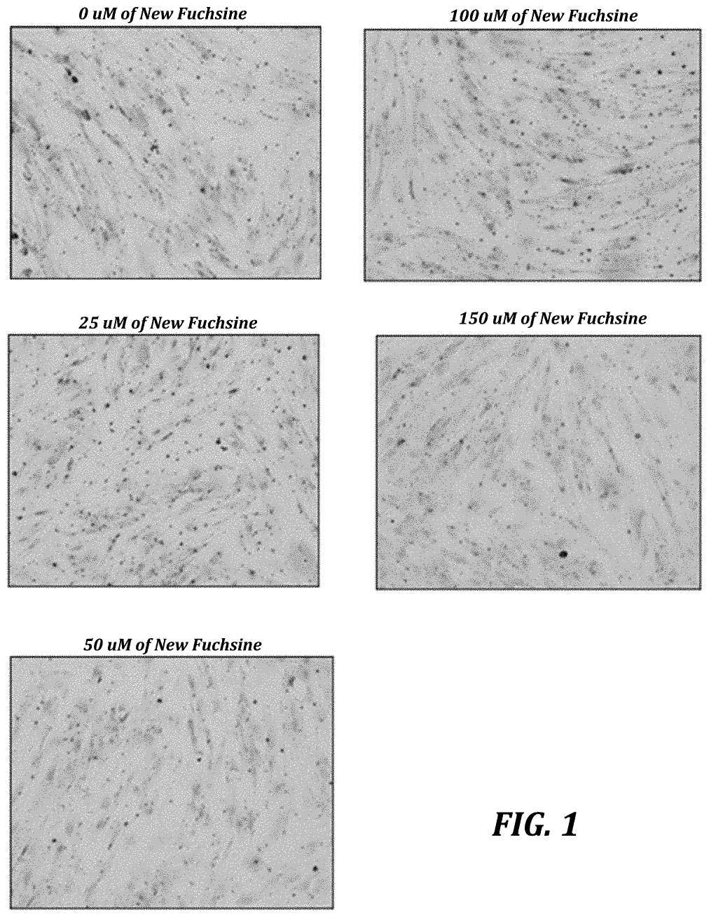

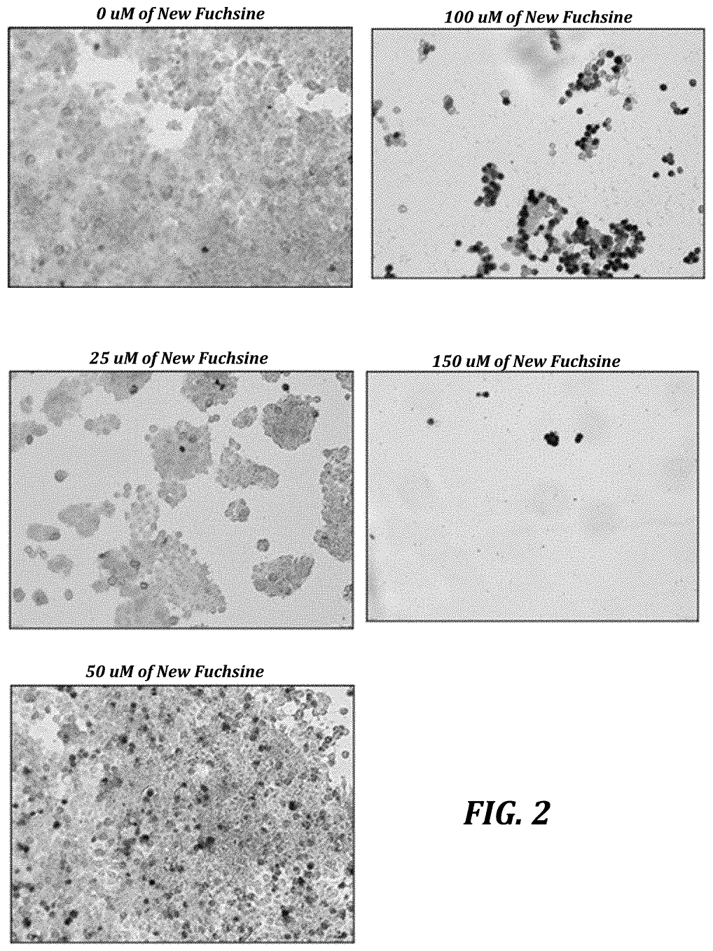

As seen in FIGS. 1 and 2, new fuchsine in all tested concentration did not exhibit any visual toxicity to normal human epithelial like cells (CRL-1790). First signs of toxicity to colon carcinoma cells (HT29) can be seen at the concentration of new fuchsine 50 uM. On the concentration of new fuchsine 150 uM it has 100% toxicity to carcinoma cells which corresponds to empty plastic.

### 2. New Fuchsine Treatment of Human Fibrosarcoma Cells (HT-1080) vs Normal Human Fibroblasts (CRL-1790)

In this second Example, the effect of treatment by different concentration of New Fuchsin on normal human epithelial like cell line CRL 1790 (ATCC) and Human fibrosarcoma cell line (HT1080) was studied.

The general study methods of study were as follows:

2 flasks Thermo Scientific Nunc Nunclon Delta Surface 12.5 cm (Cat. No 136196) with McCoy's5A complete medium were seeded with cell culture of CRL1790 (ATCC) and 2 flasks Thermo Scientific Nunc Nunclon Delta Surface 12.5 cm (Cat. No 136196) with DMEM complete medium were seeded with cell culture of HT1080 (ATCC) and were kept at 37 degrees 5% CO2 in CO2 incubator until it reached of 100% confluence (2 days).

After reaching of 100% confluence medium with 10% of FBS was changed to the same medium with different concertation of New Fuchsin (0 uM, and 50 uM) (Sigma Cat. No 72200). Flasks were kept in the CO2 incubator 5% CO2, 37-degree C for additional 12 hours. After 12 hours of treatment all flasks were stained by fixed by 10% Formaldehyde and stained by H&E.

After completing of the staining protocol the flasks were examined in the light microscope under magnification x100 and multiple pictures were taken using Olympus BX51 microscope and Olympus DP73 digital camera.

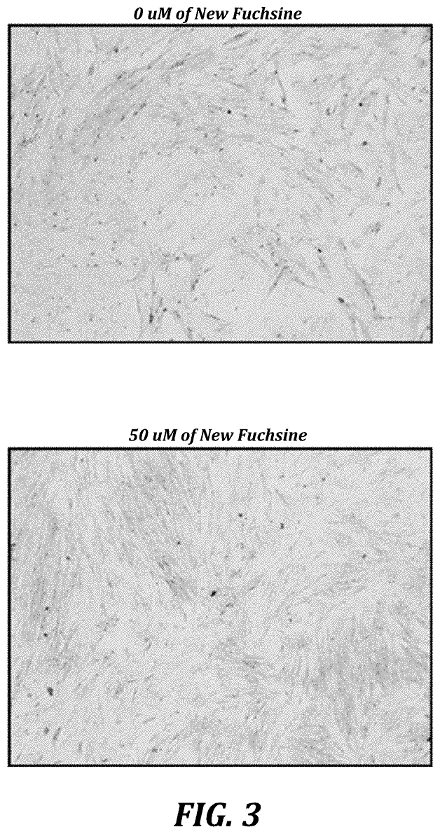

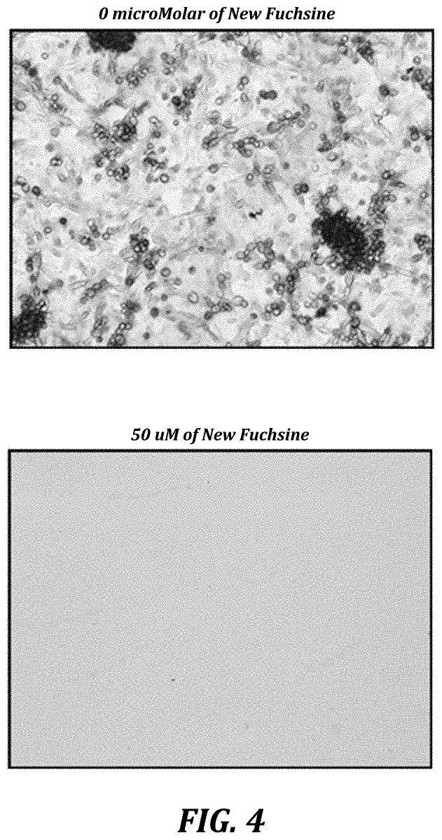

As shown in FIGS. 3 and 4, new fuchsine in all tested concentration did not exhibit any visual toxicity to normal human epithelial like cells (CRL-1790). On the concentration of new fuchsine 50 uM it has 100% toxicity to Fibrosarcoma cells (HT-1080) which corresponds to empty plastic.

### 3. New Fuchsine Treatment of Human Cervical Carcinoma Cells (HELA)

Here the effect of new fuchsine treatment of Human cervical carcinoma cells was evaluated. In this Example, different concentration of new fuchsine on Human Cervical carcinoma cell line (HeLa) was studied.

The general study methods of study were as follows:

Hela cells were growing in incubator at 37 degrees C. and 5% CO2 until 70% confluence was reached. After reaching of 70% confluence, medium was changed to medium with different concentrations of new fuchsine (0 μM, 15 μM, 31 μM, 62 μM, 125 μM and 250 μM). Flasks were kept in the CO2 incubator (5% CO2, 37-degree C) for additional 24 hours. After 24 hours of treatment, cells were detached with Trypsin, washed with fresh medium and stained with Trypan blue. Number of cells and viability were examined in automatic cell counter (Invitrogen).

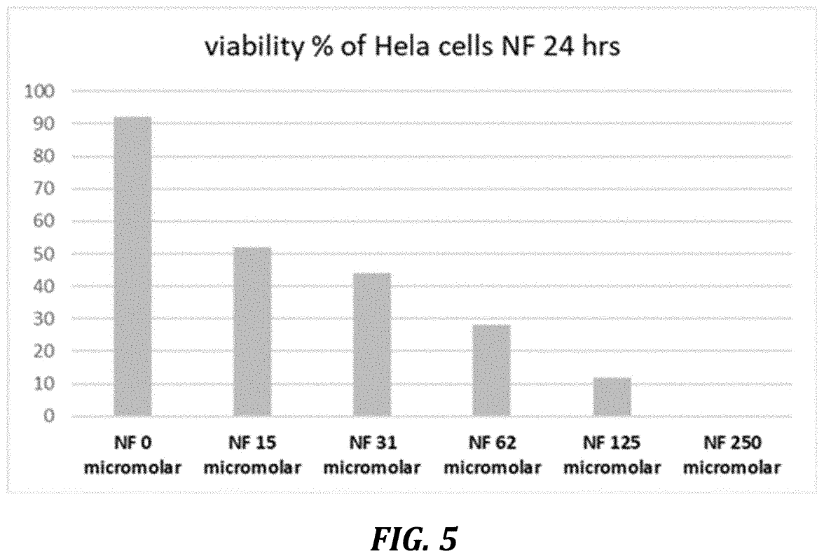

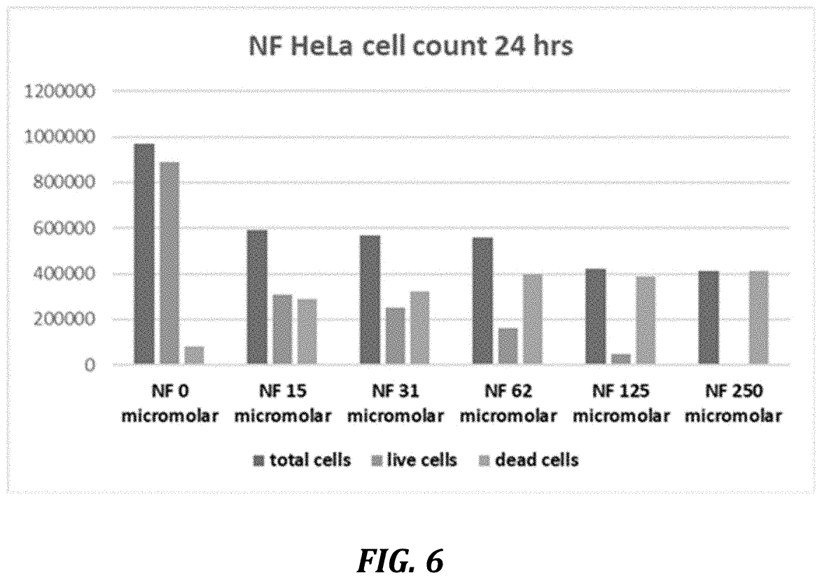

As seen in FIGS. 5 and 6, new fuchsine in all tested concentrations exhibited toxicity to human carcinoma cells. At the concentration of new fuchsine 250 uM it has 100% toxicity to carcinoma cells.

### 4. New Fuchsine Treatment of Colon Adenocarcinoma Cells (HT29)

In this Example, the effect of treatment by different concentration of New Fuchsin on human colon adenocarcinoma cells (HT29) was studied.

The general study methods of study were as follows: HT29 cells were grown in incubator at 37 degrees C. and 5% CO2 until 70% confluence was reached. After reaching of 70% confluence, medium was changed to medium with different concentration of new fuchsine (0 μM, 15 μM, 31 μM, 62 μM, 125 μM and 250 μM). Flasks were kept in the CO2 incubator (5% CO2, 37-degree C) for additional 24 hours. After 24 hours of treatment cells were detached with Trypsin, washed with fresh medium and stained with Trypan blue. Cell viability was examined in automatic cell counter (Invitrogen).

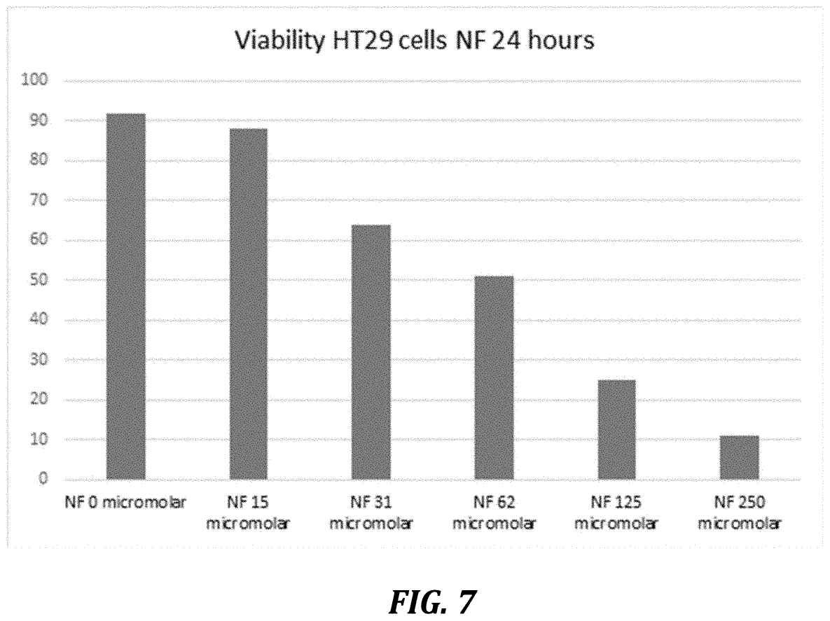

As shown in FIG. 7, new fuchsine in all tested concentration did exhibit toxicity to human colon adenocarcinoma cells (HT29), but was more effective against cervical carcinoma cells. At the concentration of new fuchsine 250 uM. 100% toxicity to colon adenocarcinoma cells was not able to be achieved.

Without wishing to be bound by a particular theory, the results were expected to demonstrate a pan tumor cytotoxic effect of new fuchsine due to its believed activity through the charges of the molecule of new fuchsine and net charges of proteins in cancer cells because of intracellular alkaline pH in cancer. While this may be true in some aspects, it was surprisingly found that new fuchsine has different cytotoxic and cytostatic activity across different kinds of malignant neoplasia, and thus, a significant difference in efficacy between different kinds of malignant tumors. As seen in the examples, it was surprising found that new fuchsine shows high efficacy, dose dependent cytotoxic effect on cervical carcinoma (HeLa cells) and was found to be much less effective for colon carcinoma (HT29 cells).

The present disclosure includes at least the following aspects: Aspect 1: A method for treating a disorder of uncontrolled cellular proliferation in a cell of a subject, the method comprising the step of providing to the cell an effective amount of at least one fuchsine compound or a pharmaceutically acceptable salt thereof, thereby treating the cell from the disorder of uncontrolled cellular proliferation. Aspect 2: The method of aspect 1, wherein the fuchsine compound is new fuchsine. Aspect 3: The method of aspect 1, wherein the at least one fuchsine compound is new fuchsine, having the structure represented by the formula:

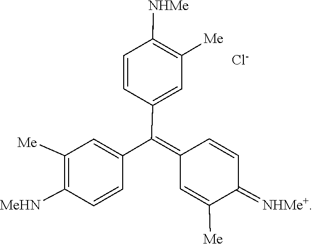

Aspect 4: The method of aspect 1 or 2, wherein the disorder of uncontrolled cellular proliferation is characterized by apoptosis, proliferation, transformative ability, gene expression profiling, or a dominant negative effect, or combinations thereof. Aspect 5: The method of aspect 1 or 4, wherein the disorder of uncontrolled cellular proliferation is a hyperproliferative disorder. Aspect 6: The method of aspect 5, wherein the hyperproliferative disorder is selected from a malignant, pre-malignant or non-malignant neoplastic disorder, inflammation, an autoimmune disorder, a hematological disorder, a skin disorder, a virally-induced hyperproliferative disorder, a myelodysplastic disorder or a myeloproliferative disorder. Aspect 7: The method of aspect 5, wherein the hyperproliferative disorder is selected from cancer, benign tumors, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

Aspect 8: The method of aspect 1 or 2, wherein the disorder of uncontrolled cellular proliferation is a cancer. Aspect 9: The method of aspect 8, wherein the cancer is a squamous cell carcinoma. Aspect 10: The method of aspect 9, wherein the squamous cell carcinoma is a metastatic squamous cell carcinoma. Aspect 11: The method of aspect 9, wherein the squamous cell carcinoma is a non-small cell lung carcinoma. Aspect 12: The method of aspect 8, wherein the cancer is a uterine carcinosarcoma. Aspect 13: The method of aspect 8, wherein the cancer is an embryonal rhabdomyosarcoma, glioblastoma, medullablastoma, or osteosarcoma. Aspect 14: The method of aspect 8, wherein the cancer is an adrenocortical tumor. Aspect 15: The method of aspect 8, wherein the cancer is a solid tumor. Aspect 16: The method of aspect 8, wherein the cancer is a hematological malignancy. Aspect 17: The method of aspect 16, wherein the hematological malignancy is a leukemia, lymphoma, or myeloma. Aspect 18: The method of aspect 17, wherein the lymphoma is multiple lymphoma or B-cell non-Hodgkin's lymphoma. Aspect 19: The method of aspect 17, wherein the myeloma is multiple myeloma. Aspect 20: The method of aspect 17, wherein the leukemia is acute myeloid leukemia. Aspect 21: The method of aspect 8, wherein the cancer is a cancer of lung, cervix, ovary, uterus, esophagus, prostate, or head and neck. Aspect 22: The method of aspect 8, wherein the cancer is a cancer of the cervix. Aspect 23: The method of aspect 21, wherein the lung cancer is a non-small cell lung cancer. Aspect 24: The method of aspect 23, wherein the non-small cell lung cancer is a squamous cell carcinoma, adenocarcinoma, or large cell-undifferentiated carcinoma. Aspect 25: The method of aspect 1, wherein the cell is a mammalian cell. Aspect 26: The method of aspect 25, wherein the mammalian cell is a human cell. Aspect 27: The method of aspect 1, wherein the effective amount is a therapeutically effective amount. Aspect 28: The method of aspect 1, wherein the effective amount is a prophylactically effective amount. Aspect 29: The method of aspect 1, further comprising the step of identifying a cell in need of anticancer treatment. Aspect 30: The method of aspect 1, wherein the effective amount of the fuchsine compound is from about 0.001 to about 10000 μM in serum medium.

Aspect 31: A method for treating a disorder of uncontrolled cellular proliferation activity in a subject, the method comprising the step of providing to the subject an effective amount of at least one fuchsine compound or a pharmaceutically acceptable salt thereof

Aspect 32: The method of aspect 31, wherein the subject has been diagnosed with a need for anticancer treatment prior to the administering step. Aspect 33: The method of aspect 31 or 32, wherein the subject is a mammal. Aspect 34: The method of aspect 33, wherein the mammal is a human. Aspect 35: The method of any of aspects 31-34, wherein the effective amount is a therapeutically effective amount. Aspect 36: The method of any of aspects 31-34, wherein the effective amount is a prophylactically effective amount. Aspect 37: The method of any of aspects 31-36, further comprising the step of identifying a subject in need of cellular hyperproliferative treatment. Aspect 38: The method of any of aspects 31-37, wherein the disorder of uncontrolled cellular proliferation is characterized by apoptosis, proliferation, transformative ability, gene expression profiling, or a dominant negative effect, or combinations thereof. Aspect 39: The method of any of aspects 31-38, wherein the disorder of uncontrolled cellular proliferation is a cancer. Aspect 40: A method for the treatment of a subject, the method comprising the steps of: diagnosing the subject as having an hyperproliferative disorder or disease; and administering to the subject an effective amount of at least one fuchsine compound or a pharmaceutically acceptable salt thereof. Aspect 41: The method of aspect 40, wherein diagnosing comprises performing a physical examination upon the subject and making a finding. Aspect 42: The method of aspect 40, wherein the hyperproliferative disorder or disease is selected from a malignant, pre-malignant or non-malignant neoplastic disorder, inflammation, an autoimmune disorder, a hematological disorder, a skin disorder, a virally-induced hyperproliferative disorder, a myelodysplastic disorder or a myeloproliferative disorder. Aspect 43: The method of aspect 40, wherein the hyperproliferative disorder or disease is selected from cancer, benign tumors, psoriatic arthritis, rheumatoid arthritis, inflammatory bowel disease, psoriasis, Reiter's syndrome, pityriasis rubra pilaris, hyperproliferative variants of the disorders of keratinization, restenosis, diabetic nephropathy, thyroid hyperplasia, Grave's Disease, benign prostatic hypertrophy, Li-Fraumenti syndrome, diabetic retinopathy, peripheral vascular disease, cervical carcinoma-in-situ, familial intestinal polyposes, oral leukoplasias, histiocytoses, keloids, hemangiomas, hyperproliferative arterial stenosis, inflammatory arthritis, hyperkeratoses, papulosquamous eruptions including arthritis, warts, and EBV-induced disease, scar formation, multiple sclerosis, systemic lupus erythematosus (SLE; lupus), myasthenia gravis, non-malignant hyperplasis, agranuloma, MGUS (Monoclonal Gammopathy of Unknown Significance, neoplastic meningitis, polycythemia vera, scleromyxedema, papular mucinosis, amyloidosis and Wegener's granulomatosis.

Aspect 44: The method of aspect 40, wherein the hyperproliferative disorder or disease is a cancer.

Aspect 45: The method of aspect 44, wherein the cancer is selected from a hematological cancer, a cancer of the breast, lung, ovary, prostate, head, neck, kidney, head, neck, pancreas, brain, ovary, kidney, prostate, breast, lung, colon, and liver.

Aspect 46: The method of aspect 45, wherein the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

Aspect 47: The method of aspect 40, wherein the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. Aspect 48: The method of aspect 40, wherein the effective amount of the fuchsine compound is from about 1 mg to about 100 mg.

Aspect 49: A pharmaceutical composition comprising an effective amount of at least one fuchsine compound or a pharmaceutically acceptable salt thereof. Aspect 50: A pharmaceutical composition comprising an effective amount of at least one fuchsine compound or a pharmaceutically acceptable salt thereof; and at least one diazo dye. Aspect 51: The composition of aspect 49, wherein the at least one diazo dye is Bismarck brown Y. Aspect 52: The composition of aspect 49 or 50, wherein the effective amount is a therapeutically effective amount. Aspect 53: The composition of aspect 49 or 50, wherein the effective amount is a prophylactically effective amount. Aspect 54: The composition of aspect 49 or 50, wherein the composition is formulated for oral administration. Aspect 55: The composition of aspect 49 or 50, wherein the composition is formulated for topical administration.

Aspect 57: A kit comprising an effective amount of at least one fuchsine compound or a pharmaceutically acceptable salt thereof, and one or more of: at least one agent known to treat a disorder of uncontrolled cellular proliferation; instructions for treating the disorder of uncontrolled cellular proliferation; and instructions for administering the fuchsine compound in connection with the disorder of uncontrolled cellular proliferation. Aspect 58: The kit of aspect 57, wherein each dose of the fuchsine compound and the agent are co-packaged. Aspect 59: The kit of aspect 57, wherein each dose of the fuchsine compound and the at least one agent are co-formulated. Aspect 60: The kit of aspect 57, wherein the dosage forms are formulated for oral administration, inhalation administration, topical administration, and/or parenteral administration. Aspect 61: The method of aspect 1 or 2, wherein the effective amount of the fuchsine compound is from about 0.001 to about 1000 μM in serum medium. Aspect 62: The method of aspect 1 or 2, wherein the effective amount of the fuchsine compound is from about 0.001 to about 250 μM in serum medium. Aspect 63: The method of aspect 1 or 2, wherein the effective amount of the fuchsine compound is from about 0.001 to about 100 μM in serum medium.

Aspect 64: The method, composition, or kit of any preceding claim, wherein the effective amount of the fuchsine compound is from about 1 mg to about 50 mg. Aspect 65: The method, composition, or kit of any preceding claim, wherein the effective amount of the fuchsine compound is from about 1 mg to about 25 mg. Aspect 66: The method, composition, or kit of any preceding claim, wherein the at least one fuchsine compound is not basic fuchsine.

Aspect 67: The method or kit of any preceding claim, further comprising an effective amount of at least one diazo dye. Aspect 68: The method or kit of any preceding claim, further comprising an effective amount of Bismarck brown Y. Aspect 69: The method, composition, or kit of any preceding claim, wherein the effective amount is a therapeutically effective amount. Aspect 70: The method, composition, or kit of any preceding claim, wherein the effective amount is a prophylactically effective amount.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating a disorder of uncontrolled cellular proliferation in a cell of a subject, the method comprising the step of providing to the cell an effective amount of at least one fuchsine compound or a pharmaceutically acceptable salt thereof, thereby treating the cell from the disorder of uncontrolled cellular proliferation.

2. The method of claim 1, wherein the at least one fuchsine compound is new fuchsine, having the structure represented by the formula:

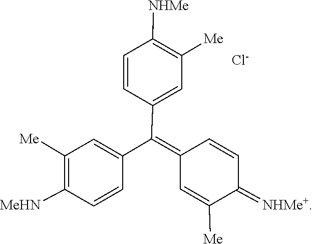

3. The method of claim 2, wherein the disorder of uncontrolled cellular proliferation is characterized by apoptosis, proliferation, transformative ability, gene expression profiling, or a dominant negative effect, or combinations thereof

4. The method of claim 3, wherein the disorder of uncontrolled cellular proliferation is a hyperproliferative disorder.

5. The method of claim 4, wherein the hyperproliferative disorder is selected from a malignant, pre-malignant or non-malignant neoplastic disorder, inflammation, an autoimmune disorder, a hematological disorder, a skin disorder, a virally-induced hyperproliferative disorder, a myelodysplastic disorder or a myeloproliferative disorder.

6. The method of claim 2, wherein the disorder of uncontrolled cellular proliferation is a cancer.

7. The method of claim 6, wherein the cancer is a squamous cell carcinoma.

8. The method of claim 6, wherein the cancer is a uterine carcinosarcoma.

9. The method of claim 6, wherein the cancer is an adrenocortical tumor.

10. The method of claim 6, wherein the cancer is a cancer of lung, cervix, ovary, uterus, esophagus, prostate, or head and neck.

11. The method of claim 10, wherein the cancer of the cervix is cervical carcinoma.

12. The method of claim 2, wherein the effective amount is a therapeutically effective amount.

13. The method of claim 12, wherein the effective amount is from about 1 mg to 100 mg.

14. The method of claim 12, wherein the effective amount is from about 0.001 to about 100 μM in serum.

15. A pharmaceutical composition comprising an effective amount of at least one fuchsine compound or a pharmaceutically acceptable salt thereof; and an effective amount of at least one diazo dye.

16. The composition of claim 15, wherein the at least one fuchsine compound is new fuchsine and the at least one diazo dye is Bismarck brown Y.

17. The composition of claim 16, wherein the effective amount is a therapeutically effective amount.

18. The composition of claim 17, wherein the composition is formulated for oral administration.

19. The composition of claim 18, wherein the effective amount is effective to be cytotoxic to cancer cells.

20. The composition of claim 18, wherein the cancer cells are cervical cancer cells.
